(19) United States
(12) Patent Application Publication
(10) Pub. No.: US 2025/0257175 A1
(43) Pub. Date: Aug. 14, 2025

(54) METHOD AND APPARATUS FOR PRETREATMENT OF A BIOMASS COMPRISING LIGNOCELLULOSIC FIBERS

(71) Applicant: CLEAN VANTAGE LLC, Richland, WA (US)

(72) Inventors: Philip TELLER, Trorod (DK); Richard GARRISON, Richland, WA (US)

(21) Appl. No.: 18/857,886

(22) PCT Filed: Apr. 13, 2023
(86) PCT No.: PCT/EP2023/059706

§ 371 Date: Oct. 18, 2024

(30) Foreign Application Priority Data
Apr. 22, 2022 (DK) PA202200370

(51) Int. Cl. C08H 8/00; B01J 8/08; C08L 97/02

(52) U.S. Cl. CPC C08H 8/00; B01J 8/085; B01J 8/087; C08L 97/02; B01J 2208/00371; C08L 2205/16

(57) ABSTRACT

Pretreatment of biomass using targeted alkaline hydrolysis with optional alkali recovery is shown, where the biomaterial is steam exploded before an alkaline hydrolysis by
a) Providing a wetted biomass,
b) Flashing the wetted biomass into a pretreatment reactor at a selected pressure,
c) Subjecting the biomass to thermal hydrolysis and steam explosion after holding the biomass at a temperature for 10-45 minutes,
d) Subjecting the steam exploded biomass to an alkaline hydrolysis by adding an alkali component in a selected concentration obtaining an alkali reacted biomass slurry.

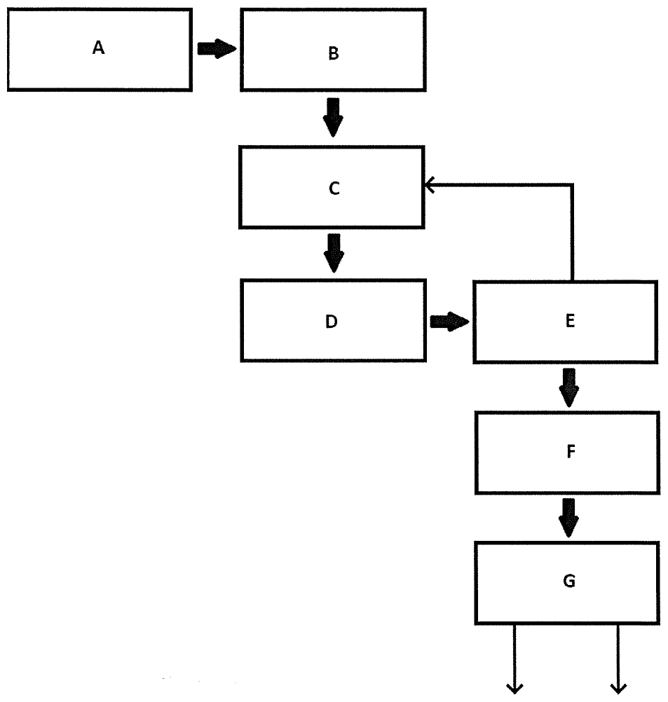

Apparatus is also shown having a conditioning unit (B), a feed sluice (C) configured to receive conditioned biomass and guide the biomass to the inlet of a pretreatment reactor/tank (D) for thermal hydrolysis/steam explosion, a flash tank (E), a hydrolysis tank (F) for alkaline hydrolysis, and a separation unit (G) in which the pretreated slurry can be separated into liquid and solid.

### TECHNICAL FIELD

The present disclosure relates generally to pretreatment of biomass using “targeted alkaline hydrolysis with optional alkali recovery”, where the biomaterial is steam exploded upfront before the alkaline hydrolysis, and where the alkali chemical is optionally recovered after the alkaline hydrolysis step. In particular, the present disclosure relates to wet hydrolysis of lignin linkages in a preconditioned biomass.

### DESCRIPTION OF RELATED ART

General definitions of “biomass” or “biomaterials” may include agricultural commodities and residues, plants and trees, algae, crop residues, waste material (including woodwaste and wood residues), animal waste and byproducts (including fats, oils, greases, and manure), construction waste, and food and yard waste. Readily available sources of biomass may include agricultural crop residues (e.g., straw, com stover, bagasse, husk, and hull etc.), purpose grown energy crops (e.g., Miscanthus and switch grass), forest residues (e.g., sawmill residues, wood chips, forest thinning's, hog fuel, and scrap wood), and wastes (e.g., municipal solid waste (“MSW”) including green waste, industrial food processing waste, manure, manure fibers, digested manure fiber and sewage sludge (“SS”).

These biomass materials may be produced in great abundance, but much of such materials may lack commercially viable end uses. In the cases of MSW and SS, great expenditures of public funds are typically used to dispose of such wastes, including costs involved in the collection, treatment, transport, and final disposal. The recovery of energy (i.e., fuels) and/or chemical products from biomass could avoid the costs of disposal as well as reduce reliance on non-renewable fossil fuel resources, which commonly serve as feedstock to produce many industrial chemicals.

However, many such biomass materials may contain lignin, hemicellulose, cellulose, and/or other like constituent components in varying amounts. Such biomass components may reduce the bio-accessibility of the biomass and be inhibitory for production of desirable fuels and other bioproducts from the biomass.

Steam explosion was introduced and patented as a biomass pretreatment process in 1926. It is a thermomechanical process to break down the structural components of cellulose. In general, steam explosion is a process in which biomass is treated with hot steam (180 to 240° C.) under pressure (1 to 3.5 MPa) followed by an explosive decompression of the biomass to atmospheric pressure that results in a rupture of the rigid structure of the biomass fibers, changing the starting material into a fibrous dispersed solid. The sudden release of pressure generates shear force which hydrolyzes the glycosidic bond and hydrogen bonds between the glucose chains. Steam explosion processes can be run in continuous or batch mode. A batch reactor is usually used for laboratory scale pretreatment while continuous systems are commonly used for large-scale, industrial processes.

### SUMMARY OF THE INVENTION

According to a first aspect of the present invention, a method is shown for pretreatment of a biomass comprising lignocellulosic fibers, comprising the following:
a) Providing a wetted biomass having a dry matter content of at least 15%, or between 15-60%, or preferably between 20-40% by weight,
b) Flashing the wetted biomass into a pretreatment reactor at a pressure between 5 and 14 bar,
c) Subjecting the biomass to thermal hydrolysis and steam explosion after holding the biomass at a temperature between 150-210° C., at steam saturation pressure between 3.7-18.5 bar, for 10-45 minutes,
d) Subjecting the steam exploded biomass to an alkaline hydrolysis by adding an alkali component in a concentration of between 0.1 to 10% w/w obtaining an alkali reacted biomass slurry.

According to a second aspect of the present invention, an apparatus is shown for pretreatment of a biomass comprising lignocellulosic fibers, the apparatus comprising
a conditioning unit (B) optionally following a filtering or pressing unit (A) configured to receive and condition e.g. downsize and/or concentrate and/or wet and/or heat the biomass to obtain dry matter content of at least 15%, or dry matter content between 15-60%, or preferably 20-40% by weight,
a feed sluice (C) configured to receive conditioned biomass and guide the biomass to the inlet of the pretreatment reactor/tank, which feed sluice (C) comprises an inlet for biomass, an outlet for pressurized biomass, one or more inlet(s)/outlet(s) for steam and a vent valve,
a pretreatment reactor/tank (D) for thermal hydrolysis and steam explosion of biomass comprising an inlet for biomass, an outlet for pretreated biomass, and one or more steam inlets,
a flash tank (E) configured to withstand high and low pressures comprising an inlet for pretreated biomass, and an outlet for flashed biomass,
a hydrolysis tank (F) in which alkaline hydrolysis takes place comprising an inlet for flashed biomass and an outlet for pretreated slurry,
a separation unit (G) in which the pretreated slurry can be separated into its liquid and solid (fiber) constituents.

In further accord with the second aspect of the invention, the flash tank (E) comprises a return loop for steam connecting the inside of the flash tank (E) to the feed sluice (C).

These and other feature of the invention will become apparent in light of the drawing that illustrates the detailed description that follows.

### BRIEF DESCRIPTION OF THE DRAWING

The sole FIGURE shows the structure of an apparatus according to the invention for carrying out the steps of the method according to the invention by means of the corresponding elements of the apparatus.

### DETAILED DESCRIPTION

In the following description, reference is made to exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiment may be utilized, without departing from the spirit and scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the features, structures, or characteristics may be combined in any suitable combinations and/or sub-combinations in one or more embodiments or examples.

The present invention provides a method and an apparatus for pretreatment of biomaterials using steam explosion subsequently followed by alkaline hydrolysis with optional recovery of the alkali chemical. When applying the invention, the bio-accessibility of biomass components can be enhanced. Embodiments of the present disclosure comprise a method performed on biomaterials to selectively hydrolyze the linkage within the lignin molecule as well as between lignin and other polymers in biomass, thereby increasing the bio-accessibility/digestibility of the biomass. The optional recovery and reuse of the alkali chemical may ensure that the concentration of alkali in the pretreated biomass is below inhibitory levels for biological conversion into bioproducts. Such methods may comprise pretreatment processes carried out to prepare the biomaterials for a subsequent fermentation or chemical conversion, which may result in the production of useful biofuels, biomaterials, biochemicals or other bioproducts.

The feed for the pretreatment method and apparatus of the present invention is a biomaterial comprising lignin or lignocellulosic fibers, and normally the feed is a wetted biomass. The feed can be pretreated using heat before subsequent alkaline hydrolysis with moderate concentrations of alkaline to open up the lignocellulosic structure of the biomaterials by dissolution of lignin linkages within the molecule as well as the linkages to other polymers. A method according to the invention may comprise continuous or semi-continuous processes carried out in several reaction vessels. In general, such processes start with a pretreatment of the biomass at relatively high dry biomass matter content preparing the material for the subsequent alkaline hydrolysis. Such processes may be referred to herein as a “targeted alkaline hydrolysis” process. A product of said process comprises a slurry that may be referred to herein as a “pretreated slurry.” The pretreated slurry from the targeted alkaline hydrolysis of the present disclosure can subsequently be separated into its liquid and fiber components, where the alkali chemical in the liquid can be recovered. The pretreated separated fiber from the targeted alkaline hydrolysis of the present disclosure may subsequently be hydrolyzed using enzymes to produce a sugar stream and/or may be fermented using a biochemical process to produce useful compounds such as volatile fatty acids (“VFAs”), alcohols, ketones, methane, and other hydrocarbons.

A targeted alkaline hydrolysis process can be carried out in an apparatus comprising a reaction assembly for carrying out each of the various processes disclosed herein. The reaction assembly may comprise the following units (see the sole FIGURE):
a conditioning unit (B) optionally following a filtering or pressing unit (A)
a feed sluice (C) configured to receive conditioned biomass and guide the biomass to the inlet of a pretreatment reactor/tank (D),
the pretreatment reactor/tank (D) is configured for thermal hydrolysis and steam explosion of biomass,
a flash tank (E) configured to withstand high and/or low pressures,
a hydrolysis tank (F) configured for alkaline hydrolysis of the flashed biomass material.
a separation unit (G) in which pretreated slurry can be separated into one or more fractions where one fraction is primary liquid, and a second fraction has a high content of solid material or fibers.
   The conditioning unit B may comprise several units forming an integrated system that first preconditions the biomass by down-sizing, concentrating, and or heating, to the desired temperature of at least 50° C., or between 50° C.-110° C., or preferably between 80° C. to 110° C., or more preferably between 90° C.-110° C., most preferably between 95° C.-105° C., and/or wetting to a desired dry matter concentration of e.g. at least 15%, or between 15-60%, or preferably between 18 to 50%, or more preferably between 20-40%, most preferably between 25-35%. The apparatus may comprise an upfront screw press A for wet materials where “wet material” normally defines that the material has a content of dry matter of less than 20%, a feed hopper, a feed sluice C that is fed from the feed hopper, a pretreatment reactor D, a high-pressure and low-pressure flash tank E, a hydrolysis tank F for alkaline hydrolysis, and optional separation equipment G for alkali recovery.

Preconditioned biomass may be collected in the feed hopper that may comprise a high-speed feed screw. A feed sluice inlet valve may be disposed between the feed hopper and the feed sluice and may selectively allow fluid communication between the feed hopper and the feed sluice, so that biomass can pass from the feed hopper into the feed sluice while the feed sluice valve is open. The feed sluice may comprise a pressure vessel, and the feed sluice may comprise a high-pressure steam inlet, e.g. more than one recycled steam inlet, and more than one feed sluice vent valve. The feed sluice may further comprise a steam inlet valve connected to the headspace of the pretreatment reactor, as well as a vent line connected to the fiber hopper, to reduce the pressure in the feed sluice before opening the feed sluice inlet valve.

In one embodiment the feed sluice (C) may further comprise more than one steam vent line connected to the fiber hopper (B), to more efficiently reduce the pressure in the feed sluice before opening the feed sluice inlet valve.

While the feed sluice outlet valve is open, biomass can pass from the feed sluice into the pretreatment reactor via a sluiced feed system. The feed sluice and pretreatment reactor may comprise pressure reaction vessels. In particular embodiments, the feed sluice and/or pretreatment reactor can withstand internal pressures of up to 16 bar.

The feed sluice may be smaller in volume than the pretreatment reactor. For example, embodiments of the feed sluice may comprise a volume that is 1-50%, preferably between 2 to 40%, more preferably between 4-30%, most preferably between 5-25% of the pretreatment reactor volume.

The pretreatment reactor may comprise an inlet conduit guiding biomass from the feed sluice to the pretreatment reactor, a pressure relief valve, a venting conduit comprising a venting valve, an outlet conduit leading to the flash tank, and one or more steam inlets. The venting conduit and venting valve may be positioned at or near the top of the pretreatment reactor. The pretreatment reactor may comprise one or more introduction zones, and an introduction zone may comprise a steam inlet port comprising inward-facing introduction ports, through which high pressure steam can be fed into the pretreatment reactor.

In one embodiment the pretreatment reactor/tank (D) for thermal hydrolysis comprises more than two steam inlets. Having multiple steam inlets (more than two) allow for a higher level of temperature control throughout the treatment process, resulting in an overall more efficient process, including higher yields and better process economy.

A pretreatment reactor D may comprise a horizontal reaction vessel having a length-to-diameter ratio of, as an example, 12 to 6, preferably between 12 to 4, more preferably between 12 to 3, most preferably between 12 to 2. Alternative embodiments having different dimensions, numbers of introduction zones, and/or size ratios may also carry out the processes of the invention.

The pretreatment reactor may be equipped with a mixer which may be configured in a way that facilitates consistent mixing within the pretreatment reactor, thereby preventing or mitigating biomass buildup. One purpose of the mixer may be to keep the pretreatment reactor sidewalls clear of biomass buildup. Additionally, mixing may help to move the biomass slurry through the pretreatment reactor and promote uniform process conditions.

The outlet conduit leads from the pretreatment reactor to the flash tank. The outlet conduit may comprise or be connected to a valve configured to regulate the flow of biomass slurry into the flash tank. The flash tank receives pretreated biomass from the pretreatment reactor. The flash tank may be equipped with a return loop to the recycled steam inlet valve of the feed sluice, thereby enabling part of the steam in the flash tank to be recycled back to the feed sluice.

The flash tank may be equipped with a mixer to help move the pretreated slurry through the flash tank to the alkaline hydrolysis tank. The flash tank may comprise a volume between 1% and 90%, preferably between 5% and 70%, more preferably between 8% to 50%, most preferably between 10% to 40% of the pretreatment reactor volume.

Alkali is added to the alkaline hydrolysis tank to further promote the degradation of the biomass materials. The alkali may be selected from NaOH, KOH, NH₄OH, Na₂CO₃, CaOH, CaO or from a mixture of these alkalis.

Optional recovery of the alkali chemicals may be done using a screw press or a membrane that may or may not be used in combination with water or steam injection.

In one embodiment the order of process steps (E), (F), (G) is preferably (E) to (F) to (G) for optimal process flow and process conditions, as well as chemical reuse.

In one embodiment the hydrolysis tank (F) in which alkaline hydrolysis takes place comprises at least two inlets for chemical dosage. One inlet for liquid alkali chemical, and one inlet for solid (powdered, granulated) alkali chemical.

In operation, biomass is pretreated using a targeted alkaline hydrolysis process carried out in an apparatus comprising a feed sluice, pretreatment reactor, a flash tank followed by an alkaline hydrolysis reactor resulting in the pretreated alkali reacted slurry. The pretreated slurry may optionally be separated into two or more fractions where a first fraction is rich in liquid and a second fraction is rich in fibrous components, to allow for alkali recovery and reduction of potential inhibitory effects on biological conversion of the pretreated slurry.

The pretreatment reactor may comprise a semi-continuous or continuous type reaction vessel into which biomass is fed via a high-pressure feed sluiced inlet chamber. The biomass may be preconditioned as described below prior to feeding into the pretreatment reactor. In general, steam is added to the biomass in the pretreatment reactor to effect the pretreatment process. After the pretreatment reactions are carried out, the pretreated slurry can be flashed from the pretreatment reactor into the flash tank, from where it will be flashed to the alkaline hydrolysis reactor for a second hydrolysis of the material. After alkali hydrolysis, the alkali chemical may optionally be recovered by separating the fiber and liquid.

Examples of biomass feedstock that could be processed in operations of the present disclosure include, but are not limited to manure, manure fibers, digested manure fibers, corn stover, straw, husk and other agricultural residues, grass, wood and other woody residues, municipal household waste and components hereof such as food waste and green waste and slaughter-house waste. In general, pretreatment of biomass may be more effective if the biomass has been reduced in size. Biomass size reduction may be carried out by using commonly available size reduction equipment, such as a chipper, shredder, or grinder to yield a size reduced biomass. A size reduced biomass may be no larger than approximately four (4) inches (101.6 mm), preferably no larger than three (3) inches (76.2 mm), more preferably no larger than two (2) inches (50.6 mm), most preferably no larger than one (1) inch (25.3 mm) in size. In alternative embodiments, other biomass feedstock sizes may be used.

The biomass may be preconditioned prior to the pretreatment process. This preconditioning process may be carried out, for example, through one or more extraction processes where salts and/or water-soluble xylan, xylose, and/or cellulose are removed from the biomass. In general, preconditioning of the biomass may be carried out to obtain biomass fibers comprising a relatively high concentration of lignin components. The preconditioning and/or extraction step may also allow water to penetrate deep into the biomass, thus creating an ion bridge throughout the lignocellulosic structure. As a result, the lignin in the biomass may be more easily targeted for the targeted alkaline hydrolysis.

The method may also include a step for biological pre-processing using anaerobic digestion (“AD”). An AD step may be carried out to convert some of the readily available biomass components like sugar monomers, oligomers, and organic acids from the feed stock into biogas before the targeted alkaline pretreatment, which may be carried out on the solids remaining after AD. Removal of easily digestible biomass components, such as readily available sugar monomers, oligomers, and organic acids may reduce unwanted reactions that could otherwise happen if raw or untreated lignocellulosic biomass were subjected to targeted alkaline hydrolysis.

Preconditioning a biomass may comprise providing a target dry matter content in the biomass. For example, the biomass can be preconditioned in a press or filter, e.g. in a screw press to achieve a dry matter content (by weight) of above 15%, or above 18%, or alternatively 15-60%, preferably between 18 to 50%, more preferably 20-40%, most preferably between 25-35%. Optionally, heating using steam or hot water can by carried out during the pressing or filtering operation.

Biomass or preconditioned biomass may be fed into a feed hopper at ambient pressure guiding the preconditioned biomass into the feed sluice. At such operations, the feed sluice inlet valve remains open to allow passage of the biomass into the feed sluice from the feedhopper. While biomass is transferred into the feed sluice, the feed sluice outlet valve may be closed, and the feed sluice vent valve may be open. After filling, the feed sluice inlet valve and the feed sluice vent valve can be closed. The preconditioned biomass within the feed sluice can be preheated using a combination of, for example, recycled steam at the recycled steam inlet, steam from the pretreatment reactor, and/or high-pressure steam at the high-pressure steam inlet. The high-pressure steam may be at a temperature of between 110 and 260° C. at 0.42-46 bar. The recycled steam may comprise lower pressure steam recovered via a return loop from the flash tank, and/or, steam from the headspace of the pretreatment reactor to be added to the recycled steam inlet valve of the feed sluice. The feed sluice can be pressurized using high-pressure steam to pressurize the biomass materials prior to feeding into the pretreatment reactor.

The feed sluice outlet valve can be flashed open at a pressure above 0.5 bar, or at a pressure between 0.5-16 bar, preferably between 3-14 bar, more preferably between 5-11 bar, most preferably between 6-10 bar. The feed sluice outlet valve may be flashed open upon reaching a specific positive delta pressure between the feed sluice and the pretreatment reactor, this specific delta pressure can be between 0.0-8 bar, preferably between 0.05-5.0 bar, more preferably between 0.1 and 3.0 bar, most preferably between 0.2 and 2.0 bar. E.g., the feed sluice outlet valve may be open for 0.01-3600 seconds, preferably between 5-600 seconds, more preferably between 10-300 seconds, most preferably between 15-90 seconds. This action may result in a flash release of the feed sluice contents into the pretreatment reactor. The feed sluice outlet valve is then closed and the remaining pressure in the feed sluice is released through the feed sluice vent valve.

In example embodiments, the pretreatment reactor temperature is maintained above 120°, or between 120-230° C., or preferably between 135-210° C., or more preferably between 145-200° C., or most preferably between 155-190° C., at steam saturation pressure of at least 1 bar, or between 1-26.9 bar, or preferably between 2.1-18 bar, or more preferably between 3.1-14.5 bar, or most preferably between 4.4-11.5 bar. The biomass temperature may be manipulated via the introduction zones by selectively adjusting the temperature and/or amount of steam introduced, thereby forming a temperature gradient along the first part of the pretreatment reactor from 80° C. to a desired temperature of e.g. between 120-230° C., or preferably between 135-210° C., or more preferably between 145-200° C., or most preferably between 155-190° C.

The biomass may be retained within the pretreatment reactor and subjected to steam for at least 1 min, or for 1 to 60 minutes, or preferably between 5-45 minutes, or more preferably between 10-40 minutes, or most preferably between 15-30 minutes.

The steam is added to the pretreatment reactor causing thermal hydrolysis to occur as soon as the temperature reaches a critical temperature for a specific biomass compound. In these cases, thermal hydrolysis can begin as the temperature approaches a target temperature.

The pretreated biomass may be transferred to the flash-tank by opening a flash-valve connecting the flash tank to the pretreatment reactor. In this embodiment the flash-tank is operated at a lower pressure than the pretreatment reactor, thus allowing for the transfer of biomass from a higher pressure to a lower pressure.

The pretreated biomass from the flash tank may be transferred to the alkali hydrolysis tank by opening a flash-valve connecting the alkali hydrolysis tank to the flash tank. In this embodiment the alkali hydrolysis tank is operated at a lower pressure than the flash-tank, thus allowing for the transfer of biomass from a higher pressure to a lower pressure.

Alkali chemical(s) may be added to the biomass, after the pretreatment process in the pretreatment tank to make the biomass structure more accessible for biological degradation. Examples of alkali chemicals that may be added include are NaOH, CaOH, KOH, NH₄OH, Na₂CO₃, CaO or a mixture of two or more of these alkali chemicals at concentrations from 0.1 to 20%, preferably between 0.25 to 10%, more preferably between 0.5-5%, most preferably between 0.5-2.5%.

The alkali hydrolysis reaction temperature, i.e. the temperature inside the alkali hydrolysis reactor, may be maintained above 75°, or between 75-125° C., or preferably between 80-115° C., or more preferably between 85-110° C., or most preferably between 90-105° C. throughout the alkali hydrolysis.

The biomass may be retained within the alkali hydrolysis reactor and subjected to one or more alkali chemicals for at least 1 minute, or for 1 to 240 minutes, or preferably between 10-180 minutes, or more preferably between 15-120 minutes, or most preferably between 20-60 minutes.

The alkali chemical can optionally be partly recovered for reuse by separating the alkali reacted biomass slurry into at least two fractions, a first fraction mainly comprising liquid and second fraction having a high content of fibers, a fiber fraction.

The alkali chemical can be recovered in a pressing or filter operation, e.g. by using a screw press with optional addition of water or steam during separation. Preferably, between 30 to 90%, or preferably between 40 to 80%, or more preferably between 45-80%, or most preferably between 60-80% of the alkali chemical may be recovered. E.g., at least 20%, at least 30%, at least 40%, at least 50%, at least 60%, at least 70%, at least 80%, at least 90%, or at least 95% of the alkali chemical may be recovered using the screw press.

The alkali chemical from the alkali hydrolysis is preferably at least partly removed before adding the fibers i.e. the solid fraction after separation to a process of biological conversion into biofuels, biochemicals or other bio-products. Normally, the solid fraction comprises approximately 30% dry-matter and 70% water (liquid) but this may vary.

After biomass is introduced into the pretreatment reactor, the biomass may travel through the pretreatment reactor in a plug flow manner. The biomass can be flashed out through the outlet conduit leading to the flash tank as pretreated biomass. Steam in the flash tank can be recycled back through the system via the return loop from the flash tank to the recycled steam inlet valve of the feed sluice. Remaining steam pressure can transport the pretreated slurry downstream from the flash tank to the alkali hydrolysis tank. The process may then begin again with new preconditioned biomaterial entering the feed sluice.

As will be understood by those skilled in the art having the methods and apparatus set forth herein may present several advantages over other methods of pretreating biomaterials. For example, embodiments of the present disclosure comprise a continuous process. Accordingly, the pretreatment reactor need not undergo significant temperature and pressure fluctuations, as may be associated with a batch type operation.

Additionally, due to the high dry matter content of the preconditioned biomass, a process according to the present invention may consume less energy to heat the reaction mixture and may have less demanding equipment needs, thus reducing operational costs. As another benefit of some embodiments is that no base or acids are present in the high temperature pretreatment reactor resulting in less corrosive pretreatment conditions, thus prolonging the lifespan of equipment, seals and apparatus employed to carry out the pretreatment processes.

Although the present disclosure is described in terms of certain preferred embodiments, other embodiments will be apparent to those of ordinary skill in the art, given the benefit of this disclosure, including embodiments that do not provide all the benefits and features set forth herein, which are also within the scope of this disclosure. It is to be understood that other embodiments may be utilized, without departing from the spirit and scope of the present disclosure.

1. A method for pretreatment of a biomass comprising lignocellulosic fibers, the method comprising:
a) Providing a wetted biomass having a dry matter content of at least 15%, or between 15-60%, or preferably between 20-40% by weight,
b) Flashing the wetted biomass into a pretreatment reactor at a pressure between 5 and 14 bar,
c) Subjecting the biomass to thermal hydrolysis and steam explosion after holding the biomass at a temperature between 150-210° C., at steam saturation pressure between 3.7-18.5 bar, for 10-45 minutes, and
d) Subjecting the steam exploded biomass to an alkaline hydrolysis by adding an alkali component in a concentration of between 0.1 to 10% w/w obtaining an alkali reacted biomass slurry.

2. The method according to claim 1, wherein the alkali component is selected from NaOH, CaOH, KOH, NH₄OH, Na₂CO₃, and CaO or a combination of one or more of these alkali components.

3. The method according to claim 1, wherein step d) is performed at a temperature between 75-120° C., preferably between 80-115° C., or preferably between 85-110° C.

4. The method according to claim 1, wherein the holding period in step d) is at least 10 minutes, or between 10-240 minutes, or preferably between 10-180 minutes, or preferably between 15-120 minutes, or preferably between 20-60 minutes.

5. The method according to claim 1, wherein the alkali reacted biomass slurry obtained at step d) is separated into at least two fractions, a first fraction having a high liquid content and a second fraction having a high content of solids.

6. The method according to claim 5, wherein the added alkali chemical is at least partly recovered from the first fraction.

7. The method method according to claim 6, wherein the alkali chemical recovered from the first fraction is reused in step d).

8. The method according to claim 1, wherein the biomass is selected from agricultural residues such as straw, husk or hulls, woody materials including woody waste products such as sawdust, waste such as household waste, food and green waste, paper pulp, slaughterhouse waste, manure, manure fibers, and manure fibers after anaerobic digestion.

9. An apparatus for pretreatment of a biomass comprising lignocellulosic fibers, the apparatus comprising
a conditioning unit (B) optionally following a filtering or pressing unit (A) configured to receive and condition e.g. downsize and/or concentrate and/or wet and/or heat the biomass to obtain dry matter content of at least 15%, or dry matter content between 15-60%, or preferably 20-40% by weight,
a feed sluice (C) configured to receive conditioned biomass and guide the biomass to the inlet of the pretreatment reactor/tank, which feed sluice (C) comprises an inlet for biomass, an outlet for pressurized biomass, one or more inlet(s)/outlet(s) for steam and a vent valve,
a pretreatment reactor/tank (D) for thermal hydrolysis and steam explosion of biomass comprising an inlet for biomass, an outlet for pretreated biomass, and one or more steam inlets,
a flash tank (E) configured to withstand high and low pressures comprising an inlet for pretreated biomass, and an outlet for flashed biomass,
a hydrolysis tank (F) in which alkaline hydrolysis takes place comprising an inlet for flashed biomass and an outlet for pretreated slurry, and
a separation unit (G) in which the pretreated slurry can be separated into its liquid and solid (fiber) constituents.

10. The apparatus according to claim 9, wherein the pretreatment reactor/tank (D) comprises more than two steam inlets.

11. The apparatus according to claim 9, wherein the combination of the following units (E, F, G) define a sequence of process steps being arranged in this exact order where material flows from the first to the third unit:
First—the flash tank (E) configured to withstand high and low pressures comprising an inlet for pretreated and/or steam exploded biomass, and an outlet for pretreated and/or steam exploded biomass,
Second—the hydrolysis tank (F) in which alkaline hydrolysis takes place comprising an inlet for pretreated and/or steam exploded biomass and an outlet for alkali treated pretreated and/or steam exploded biomass slurry,
Third—the separation unit (G) in which the alkali treated pretreated biomass slurry can be separated into its liquid and solid (fiber) constituents.

12. The apparatus according to claim 9, wherein the hydrolysis tank (F) in which alkaline hydrolysis takes place comprises at least two inlets for chemical dosage, one for liquid chemical dosage, and a second for solid (powered, granulated) chemical dosage.

13. The apparatus according to claim 9, wherein the flash tank (E) comprises a return loop for steam connecting the inside of the flash tank (E) to the feed sluice (C).

14. The apparatus according to claim 9, wherein the conditioning unit (B) is connected to the feed sluice (C) with more than one steam pipe connection, where steam pressure can be rapidly released from the feed sluice (C) into the conditioning unit (B) before opening the biomass inlet port of the feed sluice (C).

15. The apparatus according to claim 9, wherein the pretreatment reactor (D) is connected to the feed sluice (C) with a steam pipe connection, where steam pressure can be rapidly released from the reactor (D) into the feed sluice (C).
